(19) United States
(12) Patent Application Publication
(10) Pub. No.: US 2018/0334763 A1
(43) Pub. Date: Nov. 22, 2018

(54) BREAST SAVER

(71) Applicant: Frank Liguori, Lindenhurst, NY (US)

(72) Inventor: Frank Liguori, Lindenhurst, NY (US)

(21) Appl. No.: 15/600,718

(22) Filed: May 20, 2017

(51) Int. Cl. D03D 1/00; A41D 13/008; G21F 3/02; A41D 13/05; A61N 1/16

(52) U.S. Cl. CPC D03D 1/0058; A41D 13/008; G21F 3/02; A61N 2005/1094; A61N 1/16; A41D 2400/00; A41D 13/05

(57) ABSTRACT

A breast saver is described that has the structure of a brassiere. The brassiere includes a first cup, a second cup, a first wing and a second wing. The first cup and the second cup include an electromagnetic shielding layer that is an elastic layer that retains the level of electromagnetic shielding in at least one direction of stretching. The first cup connects to the first wing and the second cup connects to the second wing. The wings of the breast saver can also include an electromagnetic shielding layer. The shielding provided by the electromagnetic shielding layer can vary in structure and material in order to provide a desired level of electromagnetic shielding while stretching in one or more directions.

### BACKGROUND OF THE INVENTION

### Field of the Invention

The present disclosure relates to the field of brassieres and more particularly to an apparatus and method for a brassiere that provides breasts with protection from many forms of electromagnetic radiation.

### Description of the Related Art

Materials that provide shielding for electromagnetic radiation are well known and have been integrated into clothing for industrial purposes for an extended period. The environment in which we live has evolved, however, such that many people carry and use electronic devices in their daily lives that emit electromagnetic radiation. That intimacy with electronic devices has introduced increased levels of electromagnetic radiation into our bodies each day.

This disclosure is constructed to describe intimate wear, in the form of a brassiere, that reduces the exposure of the breasts to electromagnetic radiation. The cumulative dosage of daily exposure to lower levels of electromagnetic radiation have been shown in studies to create health risks. In particular, the extended exposure to external sources of frequencies high than 10 Hz can create serious health problems. It is also understood that, higher frequencies can stress and create ill health effects on the body's natural electromagnetic field. For example, higher energy waves can destroy chemical and molecular bonds creating chaos in our biochemical structures. Specifically, cell tower radiation has been linked to headaches, memory loss, low sperm count, cancer, birth defects, heart conditions and Alzheimer's.

A brassiere is need that provides a high level of electromagnetic shielding that can stretch for ease of comfort while providing the necessary support.

### SUMMARY OF THE INVENTION

A brassiere that is a breast saver from electromagnetic radiation includes a first cup, a second cup, a first wing and a second wing. The first cup and second cup include a shielding layer that is structured to be a protective shield against electromagnetic radiation. The layer of electromagnetic shielding or shielding layer reduces the electromagnetic field in a space by blocking the electromagnetic field with a barrier made of conductive and/or magnetic material. Shielding layers are typically applied to isolate electrical devices from external electromagnetic fields and/or to contain the electromagnetic field emitted by the electrical device.

Electromagnetic shielding can reduce the coupling of radio waves, electromagnetic fields and electrostatic fields. The amount of reduction of the electromagnetic field depends upon factors such as the conductivity of the material used and the structure of the materials. Electromagnetic shielding is tailored for the shielding of a defined range of electromagnetic frequency.

The breast saver is described in relation to standard anatomical directions in combination with a coordinate system. The anatomical planes include a coronal plane that is aligned with the lateral directions and inferior and superior directions; a sagittal plane that is aligned with an anterior and a posterior direction and inferior and superior directions; and a transverse plane that is aligned with the lateral directions and anterior and posterior directions. As defined herein the lateral directions are aligned with axes X and X′; the superior and inferior directions are aligned with the Y and Y′ directions, respectively; and the anterior and posterior directions are aligned with the Z and Z′ axes, respectively. It is understood that the wings, cups, straps as well as the subassemblies of the breast saver are described in relation to the anatomical directions and coordinate system for explanatory purposes. It is further understood that, for example, components of the breast saver can be arranged in a variety of locations and/or angular orientations and that the anatomical directions and coordinate system provide the ability to describe their relative angular orientation to one another. In general, the breast saver is described in a position as it is being worn.

The breast saver in one preferred embodiment has an electromagnetic shielding layer that has a first level of electromagnetic shielding when un-stretched or at rest and the electromagnetic shielding layer retains the first electromagnetic shielding level when stretched simultaneously in a first direction and in a second direction, normal to the first direction.

The breast saver in one preferred embodiment has an electromagnetic shielding layer that has a first level of electromagnetic shielding when un-stretched and the electromagnetic shielding layer retains the first electromagnetic shielding level when stretched simultaneously in a first direction, such as the longitudinal direction, and stretched in a second direction, such as an anterior-posterior direction aligned that is aligned with a sagittal plane.

The breast saver combines known elastic electromagnetic protective fabrics in a unique structure as well as heretofore unknown structures of electromagnetic protective fabrics with unique electromagnetic shielding properties.

### BRIEF DESCRIPTION OF THE DRAWINGS

FIG. 1 is a frontal view of a breast saver as described in the present disclosure;

FIG. 2 is an idealized cross-sectional side view taken along lines A-A of the breast saver of FIG. 1;

FIG. 3 is an idealized cross-sectional side view, based on the cross-sectional view of FIG. 2, of a second configuration of the breast saver of FIG. 1; and

FIG. 4 is an idealized cross-sectional side view, based on the cross-sectional view of FIG. 2, of a third configuration of the breast saver of FIG. 1.

### DETAILED DESCRIPTION OF THE INVENTION

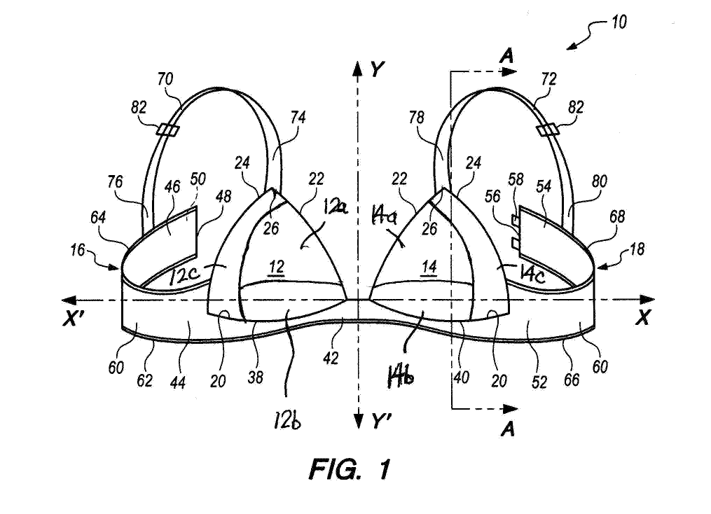

Referring initially to FIG. 1, a breast saver 10 includes a first cup 12, a second cup 14, a first wing 16 and a second wing 18. Breast saver or brassiere 10 has a traditional brassiere construction that includes first wing 16 connected to first cup 12 and second wing 18 connected to second cup 14. First wing 16 and second wing 18 preferably extend under first cup 12 and second wing 18, respectively.

First cup 12 includes a cup portion and an outer edge or a rim 38. The cup portion of first cup 12 has a flexible convex shape. First cup 12 can be designed to have any structural shape, but in this preferred embodiment approximates a tear drop shape with three sides. First cup 12 has a first edge 20, a second edge 22 and a third edge 24 that define rim 38 of first cup 12. First edge 20, second edge 22 and third edge 24 define a flexible plane that in combination with the elastic cup portion is structured to conformingly fit against a human chest and receive a human breast. First edge 20 preferably has an arcuate structure that provides conforming support the breast. First edge 20 can also include a reinforcing wire or band type element.

First edge 20 has a first terminal end located along the axes-X′ and in proximity to axes Y-Y′. In this one preferred embodiment, first edge 20 extends in a lateral direction to a second terminal end that connects with wing 18. Second edge 22, the neckline edge of first cup 12, has a first terminal end that connects to the first terminal end of first edge 20 and extends to the second terminal end that is an apex 26. Third edge 24 has a first terminal end that connects to the second terminal end of first edge 20. The second terminal end of second edge 22 and the second terminal end of third edge 24 connect to define apex 26. The second terminal ends of second edge 22 and third edge 24 connect and with first edge 20 define the continuous outer edge of first cup 12. First edge 20, second edge 22 and third edge 24 can have any shape to include, for example, arcuate or straight and can include additional edge structures. Second cup 14 is a mirror image of first cup 12. It is understood that references to first cup 12 and/or second cup 14 apply to both first cup 12 and second cup 14. For example, second cup 14 includes a rim 40 that is identical to first cup rim 38.

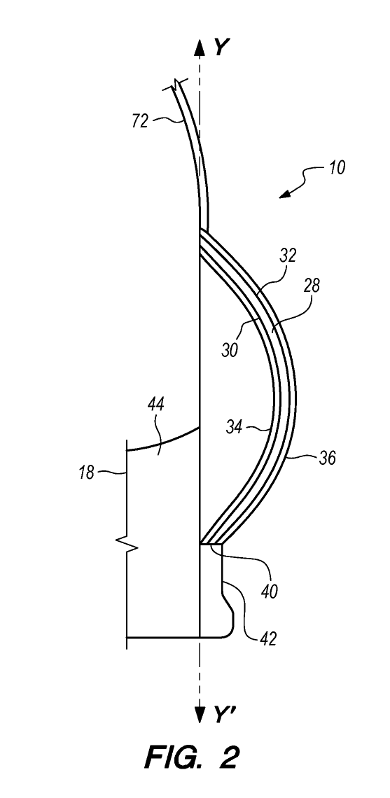

As shown in FIGS. 1 and 2, shielding layer 28 includes a first side that is an inwardly facing side 30 with a posterior direction and second side 32 that is an outwardly facing side with an anterior direction. Shielding layer 28 is structured to be an elastic protective shield against electromagnetic radiation. While brassiere 10 first cup 12 can be composed of a single layer that is shielding layer 28, first cup 12 preferably includes multiple layers of stretchable materials.

Electromagnetic shielding layer 28 or shielding layer 28 of first cup 12 is an elastic fabric with a conductive coating or plating of select fibers that provides a shield against electromagnetic radiation. The preferred material of construction of shielding layer 28 is a silver-plated nylon fiber fabric. In the preferred embodiment, the materials of layer 28 include at least 76% silver plated nylon and at least 24% elastic material. The silver is preferably at least 99% pure. The preferred materials of construction provide a 30-50 dB shielding performance in the range of 1-10 GHz. Unstretched surface resistivity of shielding layer 28 is less than 0.5 Ohm/sq. The thickness of shielding layer 28 is preferably 0.4 mm+/−0.2 mm and has a weight of 4.3 oz.+/−0.2 oz. per square yard. Shielding layer 28 preferably has a smooth nylon texture.

The preferred material for layer 28 provides equal or improved conductivity when stretched in a first direction or the opposing direction to the first direction from a position that is unstretched or at rest. The preferred material has decreased conductivity when stretched in a second direction perpendicular to the first direction. The preferred material has decreased conductivity when stretched in the second direction or a direction opposing the second direction from the unstretched position. As constructed in this one preferred embodiment of brassiere 10, electromagnetic shielding layer 28 has a stretch percentage of up to 100%+/−10% in the axes X-X′ directions and 65%+/−10% in the axes Y-Y′ directions, but brassiere 10 preferably uses a stretch percentage in the range of ten (10) to forty (40) percent (%) depending upon the intended application in and of brassiere 10. For example, it is often desirable, but not essential that the stretch percentage of first wing 16 and second wing 18 be less than that of first cup 12 and second cup 14.

In this preferred embodiment of brassiere 10, the first direction is aligned with the axes X-X′. The second direction is aligned with the axes Y-Y′. For convenience, as shown in FIG. 1 axis-X and axis-X′ have a lateral directional orientation and axis-Y and axis-Y′ have a longitudinal orientation.

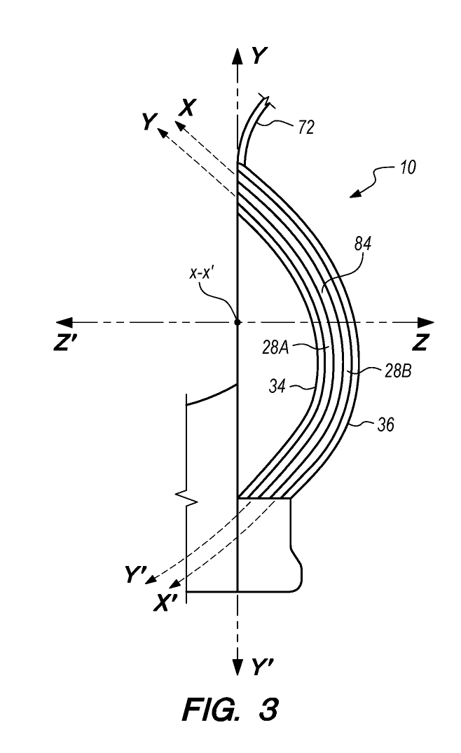

Shielding layer 28 has an inner surface 30 that is proximal or positioned in the posterior direction (as shown in FIG. 3 by axis-Z′) to shielding layer 28 and an outer surface 32 that is directed distally or in the anterior direction (as shown in Fig. by axis-Z). First cup 12 and second cup 14 can be constructed solely of shielding layer 28 or include one or more layers that interface with inner surface 30 and/or outer surface 32 of shielding layer 28. In this one preferred embodiment, a first layer 34 is in positioned adjacent inner surface 30 and a second layer 36 is positioned adjacent outer surface 32. First layer 34 and second layer 36 preferably have similar stretch, flexibility and elasticity as shielding layer 28. First layer 34 and second layer 36 preferably stretch at least 100%+/−10% in the axes X-X′ directions and 65%+/−10% in the axes Y-Y′ directions.

First layer 34 and second layer 36 are preferably identical fiber layers that can be natural or man-made textile. First layer 34 and second layer 36 can be solid layers without apertures or define apertures. In the preferred embodiment, the inwardly facing sides directed to shielding layer 28 of first layer 34 and second layer 36 define a low friction interface with shielding layer 28. In the preferred embodiment, first layer 34 and second layer 36 define a barrier that encapsulates shielding layers 28 of brassiere 10. The barrier of first layer 34 and second layer 36 can also provide a sealing encapsulation of shielding layer 28. The sealing of shielding layer 28 can be provided by the type of material of first layer 34 and second layer 36 or, for example, a coating, such as a polymer coating, for example, or a chemical or heat treatment of the inwardly facing sides of first layer 34 and second layer 36. The seal provided by first layer 34 and second layer 36 can be a hermetic seal, but is preferably constructed to restrict the migration of flaked or broken pieces of conductive material coating of shielding layer 28 from passing through first layer 34 and/or second layer 36. The sealed encapsulated shielding layer 28 may also include a material, such as a lubricant, to facilitate a low friction environment and/or the longevity of the bond of the conductive layer to the fabric. First layer 34 is preferably a soft flexible elastic material such as a nylon blend.

In the preferred embodiment of first cup 12, shielding layer 28 provide equal or improved conductivity when stretched in the first directions that are the lateral directions along axis-X or the opposing direction along axis-X′ from the unstretched or at rest position. The preferred embodiment of first cup 12 also includes shielding layer 28 providing reduced conductivity when stretched in the second directions that are the longitudinal superior and inferior directions along axis-Y and/or the opposing direction along axis-Y′ from the un-stretched or at rest position.

Shielding layer 28, first layer 34 and second layer 36 define rim 38 of first cup 12 and rim 40 of second cup 14. Rim 38 is preferably a sewn connection, but can be other forms of connection known in the art. Rim 38 can connect the terminal ends and/or folded edges of first layer 34, shielding layer 28 and second layer 36 in a hem, for example. The connection between rim 38 and first wing 16 as well as between rim 40 and second wing 18 is also preferably sewn, but the connections of brassiere 10 can include any particular type of connection that provides suitable strength and décor for a lingerie type application. When present, the edges of first layer 34 and/or second layer 36 of first cup 12 are connected at rim 38 and rim 40, respectively. In the preferred embodiment, shielding layer 28, first layer 34 and second layer 36 are solely connected along first edge 20, second edge 22 and third edge 24 of rim 38 of first cup 12.

The structure of the connection between shielding layer 28 with first layer 34 and/or second layer 36 along rim 38 of first cup 12 and their respective material compositions accommodate the independent elastic stretching in the longitudinal and lateral directions of first cup 12 between first edge 20, second edge 22 and third edge 24. The low friction interface of first layer 34 and second layer 36 with shielding layer 28 is constructed to minimizes the wear of the conductive plating of shielding layer 28 when first layer 34, shielding layer 28 and second layer 36 are stretched. The low friction interface minimizes the frictional wear and degradation of the electromagnetic protection provided by shielding layer 28.

In another preferred embodiment, first cup 12 and second cup 14 are subdivided into sections 12a, 12b and 12c and 14a, 14b and 14c, respectively. Each section has a dominant direction of stretch. The sections of first cup 12 and the sections of second up 14 are mirror images of one another. In this preferred embodiment, sections 12a and 14a both stretch laterally in the directions of axes X-X′; sections 12b and 14b stretch longitudinally in the directions of axes Y-Y′; and sections 12c and 14c stretch laterally in the directions of axes X-X′. Shielding layer 28 is cut, aligned and connected to define first cup 12 and second cup 14 to have the primary alignment that does provides equal or improved conductivity when stretched in a first direction or the opposing direction to the first direction from a position that is unstretched or at rest.

In this one preferred embodiment, shielding layer 28 of first cup 12 and second cup 14 is preferably subdivided into sections 12a, 12b, and 12c as well as 14a, 14b, and 14c. First layer 34 and second layer 34 can be subdivided as per shielding layer 28 or alternatively have a construction of a different material that can provide a seal, for example, around shielding layer 28 as per above or alternatively, select portions such as sections 12a and 12c can include an additional breathable moisture wicking layer while 12c can be a ventilation web or thin weave structure. Similarly, first layer 34 can be a sealing layer that protects the wearer from the worn or flaking particles of shielding layer 28 while second layer 36 includes one or more sections of an alternative material such as, for example, a breathable moisture wicking cotton material.

The connections between of brassiere 10 are preferably sewn connections, but it is understood that the connections can be any method of connection suitable for brassieres and/or shielding layers 28. For example, first cup 12 sections 12a, 12b and 12c as well as those of rim 38, between first cup 12 and wing 16, first cup 12 and strap 70, wing 16 and strap 70 are preferably sewn together. The connections can also include, but are not limited to hem, butt and overlapping type connections.

First cup 12 is connected to first wing 16 and second cup 14 is connected to second wing 18. In this preferred embodiment, a center gore 42 preferably connects first cup 12 and second cup 14. Center gore 42 is centrally located between first cup 12 and second cup 14. Center gore 42 can also connect to an extension of the first wing 16 and/or second wing 18 under first cup 12 and second cup 14. Center gore 42 preferably provides flexible separation between first cup 12 and second cup 16. It is understood that the structure of brassiere 10 with center gore 42 is a design element of brassiere 10 and that other alternative brassiere 10 structures such as the direct connection between first cup 12 and second cup 14 or the extension of first wing 16 and second wing 18 to define an elastic continuous loop are well known.

First wing 16 has a first end portion 44 that connects to first cup 12 and an opposing second end portion 46. First wing 16 second end portion 46 has a terminal end 48. A first connector 50 is attached to first wing 16 second end portion 46 in proximity to terminal end 48. Second wing 18 has a first end portion 52 that connects to second cup 14 and an opposing second end portion 54. Second wing 18 second end portion 54 has a terminal end 56. Second wing 18 includes a second connector 58 that is attached to second end portion 54 in proximity to terminal end 56. First connector 50 of first wing 16 and second connector 58 of second wing 18 can be selectively connected to define a continuous loop and disconnected to define an opening between first wing 16 and second wing 18. The fastening together of first connector 50 and second connector 58 defines a closed loop brassiere 10 that includes first wing 16, first cup 12, center gore 42, second cup 14 and second wing 18.

First wing 16 and second wing 18 preferably have an elastic shielding layer 60 that is the same as shielding layer 28 of first cup 12 and can further include encapsulating layers such as first layer 34 and second layer 36 as described herein. First wing 16 defines a first edge 62 and a second edge 64. First wing 16 first edge 62 and second edge 64 extend between first cup 12 and terminal end 48. Elastic shield layer 60 is defined between the inferior located first edge 62, superior located second edge 64 and terminal end 48 of first wing 16.

Second wing 18 defines a first edge 66 and a second edge 68. Second wing 18 first edge 66 and second edge 68 extend between first cup 12 and terminal end 56. Elastic shield layer 60 is defined between the inferior located first edge 66, superior located second edge 68 and terminal end 56 of second wing 18. First wing 16 and second wing 18 can vary in their distances between first edge 62 and second edge 64 and first edge 66 and second edge 68, respectively in order to encompass a greater protective surface area of shielding layer 60 depending upon the desired design of brassiere 10.

Brassiere 10 can also include a first shoulder strap 70 and a second shoulder strap 72. First shoulder strap 70 has a first end portion 74 that connects to first cup 12 and a second end portion 76 that connects to first wing 16 on or in proximity to second end portion 46. Second shoulder strap 72 has a first end portion 78 that preferably connects to second cup 14 and a second end portion 80 that preferably connects to second wing 18 on or in proximity to second end portion 54. In the preferred embodiment, first shoulder strap 70 first end portion 74 connects to apex 26 of first cup 12 and second shoulder strap 72 second end portion 78 connects to apex 26 of second cup 14.

First shoulder strap 70 and second shoulder strap 72 can vary in structure. For example, the width of the first shoulder strap 70 and second shoulder strap 72 can vary from a relatively narrow width to a wider band type structure. First shoulder strap 70 and second shoulder strap 72 are preferably structured to include an adjustment device 82 that is structured to enable the adjustment of the length of first shoulder strap 70 and second shoulder strap 72. Devices to adjust the shoulder straps of brassieres are well known in the industry and can include a variety of adjustment devices 82 such as slides, rings and/or hooks. First shoulder strap 70 and second shoulder strap 72 can also be a stretchable material as well as the same material as the electromagnetic shielding layer 28 in first cup 12 and second cup 14.

Referring now to FIGS. 1 and 3, another preferred structure for brassiere 10 includes two orthogonally aligned shielding layers 28. The directional alignment of a first shielding layer 28A provides equal or improved conductivity when stretched in the first direction that is laterally aligned with axes X and X′ from the position that is unstretched or at rest and reduced conductivity when stretched in the longitudinal directions of axes Y and Y′ as described previously. The second layer 28B has axes X and X′ aligned with the axes Y and Y′ of the first layer 28. This arrangement provides two orthogonally aligned layers 28 that in combination provide a stable or enhanced electromagnetic shielding when cup 12 and/or cup 14 is stretched in either or both the lateral X and X′ directions and the longitudinal Y and Y′ directions. The first shielding layer 28A and the second shield layer 28B in each cup 12 and 14 is preferably separated by a low friction layer 84. Layer 84 can be, for example a porous weave, webbing, mesh or other structures with or without apertures that separate and reduce the friction between the relative movement of first layer 28A and second layer 28B. Layer 84 provides separation and reduced friction when first cup 12 and second cup 14 are stretched.

Referring now to FIGS. 1 and 2, still another preferred material for layer 28 provides a single layer 28 that has equal or improved conductivity when stretched in the direction X or the opposing direction X′ from a position that is un-stretched or at rest and an equal or improved conductivity in the second direction Y or the opposing direction Y′ that is perpendicular to the first direction from the position that is un-stretched or at rest. The material for layer 28 in this embodiment has an enhanced structure that preferably has a single integrated double thickness of shielding layer 28 that accommodates a stable or improved electromagnetic protective structure that retains a sufficient density of conductive material to retain the specified protective levels when stretched in the lateral X and X′ direction and the orthogonal longitudinal Y and Y′ direction.

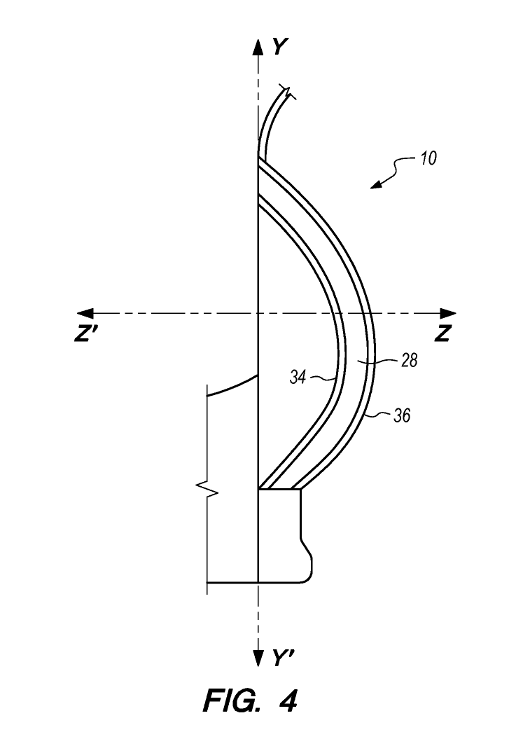

As shown in FIGS. 1 and 4, another preferred material for shielding layer 28 is located between first layer 34 and second layer 36. In this preferred embodiment of brassiere 10, shielding layer 28 provides equal or improved conductivity when stretched in the direction X or the opposing direction X′ from a position that is unstretched or at rest, a second direction Y or the opposing direction Y′ that is perpendicular to the first direction from a position that is unstretched or at rest and equal or improved conductivity when stretched in a third direction Z or an opposing direction Z′ to the third direction Z from a position that is unstretched or at rest.

The material for layer 28 in this embodiment has an enhanced three-dimensional structure with a greater thickness that accommodates a three-dimensional matrix electromagnetic protective structure that resists and/or elastically returns to the original state compression in the direction of axes Z-Z′. The three-dimensional structure retains a sufficient density of conductive material to retain the same or enhanced protective levels when stretched in three opposing orthogonal directions of the lateral X and X′ directions, longitudinal Y and Y′ and anterior-posterior Z and Z′ directions. This configuration can be three separate layers 28, but is preferably a single three-dimensional arrangement with sufficient structural weaving of protective material that provides the ability to sustain and/or improve protection when stretched in the lateral, longitudinal and anterior-posterior directions simultaneously.

Continuing with FIGS. 1 and 4, in this preferred embodiment, shielding layer 28 can also have the structure as described for FIG. 1 or the above matrix structure that is then is enclosed by a neoprene or sponge elastic low abrasion foam encapsulation that is commonly used in water sports suits and athletic wear. The stretchable foam products have reduced elasticity relative to the preferred embodiment of shielding layer 28 described above, but provide a thicker and denser encapsulation and therefore alternative first cup 12 structure. Alternative encapsulating materials for first layer 34 and second layer 36 include spacer fabrics and mesh fabrics that are breathable moisture wicking fabrics with thickness in the range of 2 mm to 5 mm. First layer 34 and second layer 36 in this configuration can provide a soft textured and preferably low friction comfort layer.

In operation as shown in FIGS. 1-4, a wearer places their arms through the first shoulder strap 70 and second shoulder strap 72 of brassiere 10 and positions their breasts in the first cup 34 and second cup 36. Brassiere 10 is then secured together using first connector 50 and second connector 58. Brassiere 10 provides the wearer with an enhanced electromagnetic shielding system for the breasts due to shielding layer 28 and the secure fit of brassiere 10 to the chest of the wearer. The structure of first cup 12 and second cup 14 provides brassiere 10 with an enclosed shielding layer 28 that is encapsulated against the undesirable breaking down of the shielding layer 28. The encapsulated shielding layer 28 can also include materials as described previously to enhance the longevity of the conductor to fiber bond and/or minimize wear on the conductor. Further, first wing 16 and second wing 18 shielding layer 60 can provide additional electromagnetic shielding under the arms and around the back of the wearer. Brassiere 10 can be adapted for athletic use as well as day to day use by the wearer.

In the preceding specification, the present disclosure has been described with reference to specific exemplary embodiments thereof. It will be evident, however, that various modifications, combinations and changes may be made thereto without departing from the broader spirit and scope of the invention as set forth in the claims that follow. While the present disclosure is described in terms of a series of embodiments and/or arrangements, the present disclosure can combine one or more novel features of the different embodiments. The specification and drawings are accordingly to be regarded in an illustrative manner rather than a restrictive sense.

What is claimed is:

1. A brassiere that includes electromagnetic shielding, the brassiere comprises:
a first cup and a second cup, the first cup and the second cup each include a cup portion and a rim, the first cup and the second cup aligned laterally;
a shielding layer included in the first cup and the second cup, the shielding layer an elastic electromagnetic shielding layer, the shielding layer retains electromagnetic shielding protection when stretched in the lateral directions;
a first layer and a second layer included in the first cup and the second cup, the first layer and second layer enclose the shielding layer, the first layer positioned proximal to the shielding layer and the second layer positioned to the shielding layer; and
a first wing that connects to the first cup and a second wing that connects to the second cup.

2. The brassiere of claim 1, wherein the shielding layer is 76% silver plated nylon and 24% elastic fiber fabric.

3. The brassiere of claim 1, wherein the shielding layer has a decreased electromagnetic protection when stretched in a longitudinal direction, the longitudinal direction normal to the lateral direction.

4. The brassiere of the claim 1, wherein the shielding layer can stretch between 10%+/−1% and 40%+/−1% in the lateral direction and 6.5%+/−1% in the longitudinal direction.

5. The brassiere of claim 1, wherein the shielding layer is at least 76% conductor plated nylon fibers and the remainder of the fibers are elastic fibers.

6. The brassiere of claim 1, wherein the silver plating of the shielding layer is at least 99.9% pure.

7. The brassiere of claim 1, wherein the brassiere includes a first shielding layer aligned to retain electromagnetic protection in the lateral directions when stretched and a second shielding layer aligned to retain electromagnetic protection in the longitudinal directions when stretched.

8. The brassiere of claim 7, wherein a layer separates the first shielding layer and the second shielding layer.

9. The brassiere of claim 1, wherein the shielding layer has a first shielding level when un-stretched and the shielding layer retains the first electromagnetic shielding level when stretched simultaneously in the lateral direction and the longitudinal direction.

10. The brassiere of claim 3, wherein the wings include a first layer, second layer and a shielding layer similar to the first cup and the second cup.

11. The brassiere of claim 1, wherein the shielding layer has a first electromagnetic shielding level when un-stretched and the shielding layer retains the first shielding layer when stretched simultaneously in the lateral direction, the longitudinal direction and an anterior-posterior direction.

12. The brassiere of claim 10, wherein the first layer, second layer and shielding layer connect at the rim of the first cup and the rim of the second cup.

13. The brassiere of claim 12, wherein the first layer, second layer and shielding layer of the first cup and the second cup are solely connected at the rim of the first cup and the rim of the second cup.

14. The brassiere of claim 1, wherein the brassiere includes a connector, the brassiere defines a loop, the connector opens and closes the loop of the brassiere.

15. The brassiere of claim 1, wherein the shielding layer includes an elastic foam and the shielding layer is encapsulated in the elastic foam.

16. The brassiere of claim 1, wherein the first layer, second layer and shielding layer have approximately identical rates of elastic stretching

17. A brassiere that includes electromagnetic shielding, the brassiere comprises:
a first cup and a second cup, the first cup and the second cup each include a cup portion and a rim, the first cup and the second cup aligned laterally;
a shielding layer included in the first cup and the second cup, the shielding layer an elastic electromagnetic shielding layer, the shielding layer a fabric layer that includes a conductive coating on at least 76% of the fiber fabric and the remainder of the fiber fabric is elastic.
a first layer and a second layer included in the first cup and the second cup, the first layer and second layer enclose the shielding layer, the first layer positioned proximal to the shielding layer and the second layer positioned distal to the shielding layer; and
a first wing that connects to the first cup and a second wing that connects to the second cup.

18. The brassiere of claim 17, wherein the shielding layer has a defined directional stretching orientation for electromagnetic shielding in which the shielding does not degrade with stretching, the shielding layer retains electromagnetic shielding protection when stretched in a first direction and a direction opposing the first direction.

19. The brassiere of claim 18, wherein the first cup and the second cup are divided into sections and each section has a defined directional stretching orientation in opposing directions, the shielding layer subdivided into sections and the directional stretching orientation of the electromagnetic shielding layer is aligned with the directional stretching orientation of the section of the first cup and the second cup.
